(19) United States
(12) Patent Application Publication
(10) Pub. No.: US 2006/0025833 A1
(43) Pub. Date: Feb. 2, 2006

(54) Variable width electrode scheme

(75) Inventor: Christopher Newton Daly, Lane Cove (AU)

Correspondence Address: JAGTIANI + GUTTAG, 10363-A DEMOCRACY LANE, FAIRFAX, VA, 22030, US

(73) Assignee: Cochlear Limited, Lane Cove (AU)

(21) Appl. No.: 11/192,014

(22) Filed: Jul. 29, 2005

Related U.S. Application Data
(60) Provisional application No. 60/591,884, filed on Jul. 29, 2004.

(51) Int. Cl. A61N 1/32

(52) U.S. Cl. 607/55

(57) ABSTRACT

In accordance with one aspect of the invention, methods and systems are disclosed for delivering a stimulating signal by a stimulating medical device having a plurality of electrodes. These methods and systems comprise electrically coupling a first set of at least two of the plurality of electrodes, and simultaneously delivering to the first set of electrically-coupled electrodes a stimulation signal suitable for application to a target tissue of a recipient.

### CROSS-REFERENCE TO RELATED APPLICATIONS

This application claims the benefit of U.S. provisional application No. 60/591,884, Jul. 29, 2004, which is hereby incorporated by reference herein in its entirety.

### BACKGROUND

1. Field of the Invention

The present invention relates generally to a stimulating medical device and, more particularly, to a variable width electrode scheme for use in stimulating medical devices.

2. Related Art

Delivery of electrical stimulation to appropriate locations within a recipient or patient (referred to herein as a recipient) may be used for a variety of purposes. For example, function electrical stimulation (FES) systems may be used to deliver electrical pulses to certain muscles of a recipient to cause a controlled movement of a limb of the recipient.

As another example, a prosthetic hearing implant system may be used to directly deliver electrical stimulation to auditory nerve fibers of a recipient's cochlea to cause the recipient's brain to perceive a hearing sensation resembling the natural hearing sensation normally delivered to the auditory nerve.

Prosthetic hearing implant systems typically have two primary components: an external component commonly referred to as a speech processor, and an implanted component commonly referred to as a receiver/stimulator unit. Traditionally, both of these components cooperate with each other together to provide sound sensations to a recipient.

The external component traditionally includes a microphone that detects sounds, such as speech and environmental sounds, a speech processor that selects and converts certain detected sounds, particularly speech, into a coded signal, a power source such as a battery, and an external transmitter antenna.

The coded signal output by the speech processor is transmitted transcutaneously to the implanted receiver/stimulator unit, commonly located within a recess of the temporal bone of the recipient. This transcutaneous transmission occurs via the external transmitter antenna which is positioned to communicate with an implanted receiver antenna disposed within the receiver/stimulator unit. This communication transmits the coded sound signal while also providing power to the implanted receiver/stimulator unit. Conventionally, this link has been in the form of a radio frequency (RF) link, but other communication and power links have been proposed and implemented with varying degrees of success.

The implanted receiver/stimulator unit traditionally includes the noted receiver antenna that receives the coded signal and power from the external component. The implanted unit also includes a stimulator that processes the coded signal and outputs an electrical stimulation signal to an intra-cochlea electrode assembly mounted to a carrier member. The electrode assembly applies the electrical stimulation directly to the auditory nerve to produce a hearing sensation corresponding to the original detected sound.

### SUMMARY

According to one aspect of the invention, methods and systems are provided for delivering a stimulating signal by a stimulating medical device having a plurality of electrodes. These methods and systems comprise electrically coupling a first set of at least two of the plurality of electrodes, and simultaneously delivering to the first set of electrically-coupled electrodes a stimulation signal suitable for application to a target tissue of a recipient.

According to another aspect, methods and systems are provided for stimulating a recipient. These methods and systems comprise disposing a plurality of tissue-stimulating electrodes in a physical arrangement on or in the recipient, and adjusting a geometry of the plurality of electrodes without replacing or altering the physical arrangement of the plurality of electrodes.

According to yet another aspect, methods and systems are provided for a cochlear implant system, comprising a plurality of electrodes disposed in a cochlear of a recipient, a speech processor for processing received acoustical signals, and a stimulator unit, responsive to the speech processor, configured to electrically couple selected electrodes and to simultaneously deliver a stimulation signal to the electrically-coupled electrodes via a stimulus current generator.

### BRIEF DESCRIPTION OF THE DRAWINGS

FIG. 1 is a block diagram of an exemplary hearing implant system suitable for implementing embodiments of the present invention.

FIG. 2 illustrates a simplified functional block diagram of a hearing implant system in accordance with one embodiment of the present invention.

FIG. 3 is a schematic block diagram of an exemplary embodiment of an output switch controller illustrated in FIG. 2.

FIG. 4 includes a series of timing diagrams illustrating the manner in which the output switch control logic illustrated in FIG. 3 controls an embodiment of output switch matrix also illustrated in FIG. 3, in accordance with embodiments of the present invention.

FIG. 5 is a schematic block diagram of an embodiment of the output switch controller illustrated in FIG. 2 that implements bi-polar stimulation.

FIG. 6A is a schematic illustration of an exemplary approach in which electrodes of the electrode array illustrated in FIG. 1 are electrically coupled to each other in accordance with one embodiment of the present invention.

FIG. 6B is a schematic illustration of an exemplary approach in which electrodes of the electrode array illustrated in FIG. 1 are electrically coupled to each other in accordance with another embodiment of the present invention.

FIG. 6C is a schematic illustration of an exemplary approach in which electrodes of the electrode array illustrated in FIG. 1 are electrically coupled to each other in accordance with a further embodiment of the present invention.

FIG. 6D is a schematic illustration of an exemplary approach in which electrodes of the electrode array illustrated in FIG. 1 are electrically coupled to each other in accordance with another embodiment of the present invention.

FIG. 7 is a graph illustrating the relative impedance of two groups of electrically-coupled electrodes and a single electrode.

FIG. 8 illustrates exemplary loudness growth functions for single, double, and triple electrode groups in accordance with one embodiment of the present invention.

FIG. 9A is a diagrammatic illustration of the current spread occurring in the auditory nerves in response to a stimulation signal conventionally applied to a single electrode on an array positioned close to the modiolus.

FIG. 9B is a diagrammatic illustration of the current spread occurring in the auditory nerves in response to a stimulation signal applied to two electrodes electrically coupled in accordance with one embodiment of the present invention.

FIG. 9C is a diagrammatic illustration of the current spread occurring in the auditory nerves in response to a stimulation signal applied to a single electrodes and two electrically-coupled electrodes on an array positioned further away from the modiolus, in accordance with one embodiment of the present invention.

FIG. 10 is a diagram illustrating how the natural increase in spread of excitation for an acoustically excited cochlea may be emulated electrically via stimulation of a group of electrodes in accordance with embodiments of the present invention.

### DETAILED DESCRIPTION

The present invention is generally directed to a stimulating medical device comprising a plurality of tissue-stimulating electrodes in which the electrode geometry may be adjusted without replacing or altering the physical arrangement of the electrodes on or implanted in a recipient. Specifically, the present invention is directed to a multi-electrode stimulating device in which a desired combination of two or more electrodes may be directly or indirectly electrically coupled to each other so that a stimulating signal may be simultaneously applied to or generated on (generally referred to as “applied to” herein) the electrically-coupled electrodes via a single source. By electrically coupling or decoupling selected electrodes in this manner, a desired electrode geometry and density may be attained.

Significantly, when implemented in a prosthetic hearing implant system, the group(s) of electrically-coupled electrodes are each managed as a single electrode along with any individual electrodes. That is, the electrode groups and single electrodes may be controlled to simultaneously or sequentially apply stimulating signals to the cochlear in accordance with a selected stimulation strategy.

Altering the electrode geometry by electrically coupling electrodes provides many advantages. Take, for example, systems in which the electrodes are arranged in a linear array, as is commonly utilized in a prosthetic hearing implant. Electrically coupling and/or de-coupling two or more adjacent or proximate electrodes of the array changes the effective electrode surface area through which a stimulating signal is applied to the auditory nerves of a cochlear. Adjusting the effective width of electrodes allows for the dynamic control of the spread of excitation by altering the region of neural excitation. In addition, the effective electrode width may be adjusted to adapt the electrode array to a cochlea having a particular pattern of functional auditory nerves.

A further advantage in the above or other applications is that electrically coupling two or more electrodes reduces electrode impedance. Because power consumption typically increases with increasing stimulus current, a reduction in electrode impedance reduces the power consumption of the implant system. This is particularly advantageous when used in conjunction with high-density electrode arrays. The design of intra-cochlea electrode arrays has been driven by the need to achieve a higher density of discrete electrodes positioned closer to the inner wall of the cochlea (or modiolus) with the objective of increasing spectral resolution and reducing stimulation thresholds. As the density of an electrode array increases (density being defined by the number of electrodes per unit length of the array), the electrode area becomes smaller, resulting in an increased impedance. By electrically coupling two or more electrodes, the impedance of the electrode array may be reduced. In addition, in hearing implant systems which utilize transcutaneous RF power/data link, the above and other benefits may be achieved without increasing RF link bandwidth utilization.

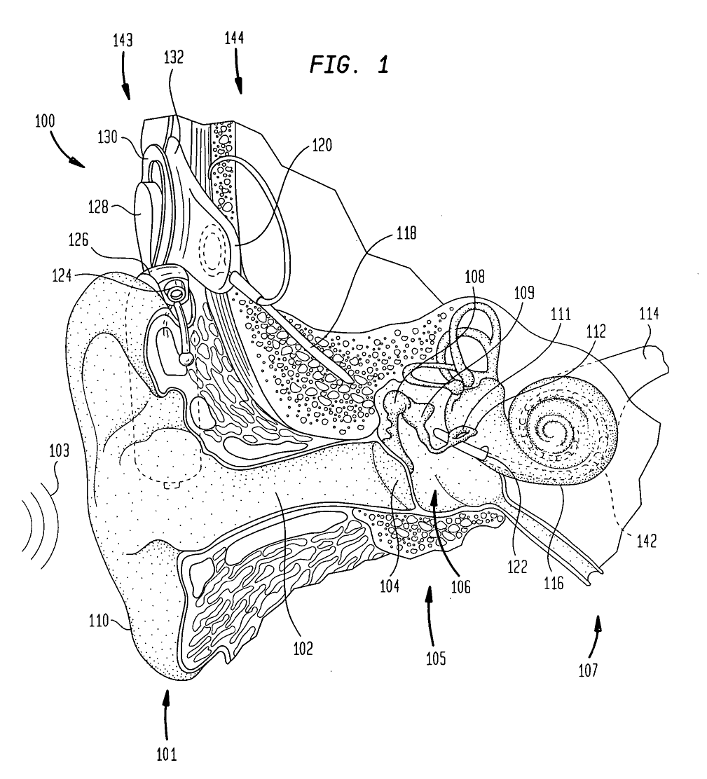

Embodiments of the present invention are described herein primarily in connection with one type of stimulating medical device, a prosthetic hearing implant system. Prosthetic hearing implant systems include but are not limited to hearing aids, auditory brain stimulators, and Cochlear™ implants (also commonly referred to as Cochlear™ prostheses, Cochlear™ devices, Cochlear™ implant devices, and the like; generally and collectively referred to as “cochlear implants” herein). Cochlear implants use direct electrical stimulation of auditory nerve cells to bypass absent or defective hair cells that normally transduce acoustic vibrations into neural activity. Such devices generally use multi-contact electrodes inserted into the scala tympani of the cochlea so that the electrodes may differentially activate auditory neurons that normally encode differential pitches of sound. Auditory brain stimulators are used to treat a smaller number of patients with bilateral degeneration of the auditory nerve. For such patients, the auditory brain stimulator provides stimulation of the cochlear nucleus in the brainstem, typically with a planar electrode array; that is, all electrode array in which the electrode contacts are disposed on a two dimensional surface that can be positioned proximal to the brainstem. FIG. 1 is a perspective view of a cochlear implant in which the effective width of the electrodes may be adjusted in accordance with tile teachings of the present invention.

Referring to FIG. 1, the relevant components of outer ear 101, middle ear 105 and inner ear 107 are described next below. In a fully functional ear outer ear 101 comprises an auricle 110 and an ear canal 102. An acoustic pressure or sound wave 103 is collected by auricle 110 and channeled into and through ear canal 102. Disposed across the distal end of ear channel 102 is a tympanic membrane 104 which vibrates in response to acoustic wave 103. This vibration is coupled to oval window or fenestra ovalis 112 through three bones of middle ear 105, collectively referred to as the ossicles 106 and comprising the malleus 108, the incus 109 and the stapes 111. Bones 108, 109 and 111 of middle ear 105 serve to filter and amplify acoustic wave 103, causing oval window 112 to articulate, or vibrate. Such vibration sets up waves of fluid motion within cochlea 116. Such fluid motion, in turn, activates tiny hair cells (not shown) that line the inside of cochlea 116. Activation of the hair cells causes appropriate nerve impulses to be transferred through the spiral ganglion cells and auditory nerve 114 to the brain (not shown), where they are perceived as sound.

Cochlear implant system 100 comprises external component assembly 143 which is directly or indirectly attached to the body of the recipient, and an internal component assembly 144 which is temporarily or permanently implanted in the recipient. External assembly 143 typically comprises microphone 124 for detecting Sound, a speech processing unit 126, a power source (not shown), and an external transmitter unit 128. External transmitter unit 128 comprises an external coil 130 and, preferably, a magnet (not shown) secured directly or indirectly to the external coil. Speech processing unit 126 processes the output of audio pickup devices 124 that are positioned, in the depicted embodiment, by ear 110 of the recipient. Speech processing unit 126 generates coded signals, referred to herein as a stimulation data signals, which are provided to external transmitter unit 128 via a cable (not shown). Speech processing unit 126 is, in this illustration, constructed and arranged so that it can fit behind the outer ear 110. Alternative versions may be worn on the body or it may be possible to provide a fully implantable system which incorporates the speech processor and/or microphone into the implanted stimulator unit.

Internal components 144 comprise an internal receiver unit 132, a stimulator unit 120, and an electrode assembly 118. Internal receiver unit 132 comprises an internal transcutaneous transfer coil (not shown), and preferably, a magnet (also not shown) fixed relative to the internal coil. Internal receiver unit 132 and stimulator unit 120 are hermetically sealed within a biocompatible housing. The internal coil receives power and data from external coil 130, as noted above. A cable or lead of electrode assembly 118 extends from stimulator unit 120 to cochlea 116 and terminates in an array 142 of electrodes. Signals generated by stimulator unit 120 are applied by the electrodes of electrode array 142 to cochlear 116, thereby stimulating the auditory nerve 114.

In one embodiment, external coil 130 transmits electrical signals to the internal coil via a radio frequency (RF) link. The internal coil is typically a wire antenna coil comprised of at least one and preferably multiple turns of electrically insulated single-strand or multi-strand platinum or gold wire. The electrical insulation of the internal coil is provided by a flexible silicone molding (not shown). In use, implantable receiver unit 132 may be positioned in a recess of the temporal bone adjacent ear 110 of the recipient.

Further details of the above and other exemplary prosthetic hearing implant systems in which the present invention may be implemented include, but are not limited to, those systems described in U.S. Pat. Nos. 4,532,930, 6,537,200, 6,565,503, 6,575,894 and 6,697,674, which are hereby incorporated by reference herein in their entireties. For example, while cochlear implant system 100 is described as having external components, in alternative embodiments, implant system 100 may be a totally implantable prosthesis. In one exemplary implementation, for example, speech processor 116, including the microphone, speech processor and/or power supply may be implemented as one or more implantable components. In one particular embodiment, the speech processor 116 may be contained within the hermetically sealed housing used for stimulator unit 126.

It should also be appreciated that although embodiments of the present invention are described herein in connection with prosthetic hearing device 100, the same or other embodiments of the present invention may be implemented in other tissue-stimulating medical devices as well. Examples of such devices include, but are not limited to, other sensory prosthetic devices, neural prosthetic devices, and functional electrical stimulation (FES) systems. In sensory prostheses, information is collected by electronic sensors and delivered directly to the nervous system by electrical stimulation of pathways in or leading to the parts of the brain that normally process a given sensory modality. Neural prostheses are clinical applications of neural control interfaces whereby information is exchanged between neural and electronic circuits. FES devices are used to directly stimulate tissue having contractile cells to produce a controlled contraction of the same. It should also be appreciated that although much of the description of the invention is directed to adjacent electrodes of an electrode array, embodiments of the present invention are not limited to adjacent electrodes or electrode arrays, but rather may be used to electrically couple any desired electrodes of a simulating medical device.

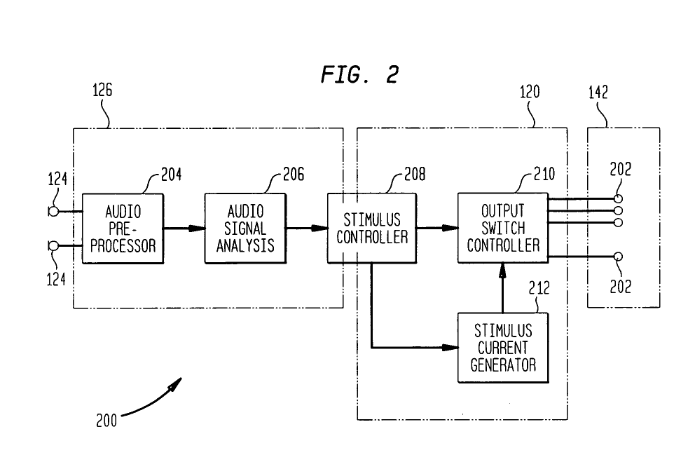

FIG. 2 is a high-level functional block diagram of one embodiment of a cochlear implant system 100, referred to herein as cochlear implant 200. The functional blocks depicted in FIG. 2 are illustrative only and may be implemented in any combination of hardware, software or combination thereof The described functions and operations may be combined as depicted in FIG. 2 or may be combined or separated as desired for a particular application.

Cochlear implant 200 comprises at least one microphone 124 as described above with reference to FIG. 1. It should be appreciated, however, that the any audio receiving device now or later developed may be implemented in a hearing prosthesis also implementing embodiments of the present invention.

Audio receiving devices 124 provide a received audio signal to an audio pre-processor 204. Audio pre-processor 204 may, for example, use a pre-emphasis filter, automatic gain control (AGC), an/or manual sensitivity control (MSC), and other signal pre-processing components. Audio-preprocessor 204 may be implemented, for example, in speech processing unit 126 described above with reference to FIG. 1. The structure and operation of audio-preprocessor 204 is considered to be well-known in the art and, therefore, is not described further herein. Further details of exemplary embodiments of audio-preprocessor 204 may be found in the U.S. patents incorporated by reference elsewhere herein in this application.

Audio pre-processor 204 provides output signals to audio signal analysis block 206. Audio signal analysis block 206 preferably filters the received signals using a bank of band-pass filters to obtain a plurality of stimulation signals and selects the maxima that will be used for stimulus application, as is well-known to those of ordinary skill in the art. Further, as is also well-known to those of ordinary skill in the art, the filter bank provides a signal for each of the stimulation channels of hearing implant 200. For example, for an implant system providing 22 channels of stimulation, the filter bank preferably outputs 22 separate signals, one corresponding to each channel of stimulation. The audio signal analysis block 206 then selects from these signals the maxima to be applied based on a stimulation strategy (e.g. 8 maxima are used) being implemented by the implant system, as is well known to those of skill in the art.

There are several speech coding strategies that may be used when converting sound into all electrical stimulation signal. Embodiments of the present invention may be used in combination with a variety of speech strategies including but not limited to Continuous Interleaved Sampling (CIS), Spectral PEAK Extraction (SPEAK), Advanced Combination Encoders (ACE), Simultaneous Analog Stimulation (SAS), MPS, Paired Pulsatile Sampler (PPS), Quadruple Pulsatile Sampler (QPS), Hybrid Analog Pulsatile (HAPs), n-of-m and HiRes™, developed by Advanced Bionics. SPEAK is a low rate strategy that may operate within the 250-500 Hz range. ACE is a combination of CIS and SPEAK. Examples of such speech strategies are described in U.S. Pat. No. 5,271,397, the entire contents and disclosures of which is hereby incorporated by reference. The present invention may also be used with other speech coding strategies, such as a low rate strategy called Spread of Excitation which is described in U.S. Provisional No. 60/557,675 entitled, “Spread Excitation and MP3 coding Number from Compass UE” filed on Mar. 31, 2004, U.S. Provisional No. 60/616,216 entitled, “Spread of Excitation And Compressed Audible Speech Coding” filed on Oct. 7, 2004, and PCT Application WO 02/17679A1, entitled “Power Efficient Electrical Stimulation,” which are hereby incorporated by reference herein. Audio signal analysis block 206 may be implemented in speech processing unit 116 of FIG. 1 by, for example, software, hardware, or any combination thereof. The structure and operation of audio-preprocessor 204 is considered to be well-known in the art and, therefore, is not described further herein. Further details of exemplary embodiments of audio-preprocessor 204 may be found in the U.S. patents incorporated by reference above and elsewhere herein in this application.

The stimulation signals may then be provided to a stimulus controller 208. Portions of stimulus controller 208 are preferably implemented in both speech processing unit 126 and the stimulator unit 120. As such, in embodiments using an external speech processing unit 126, stimulus controller 208 illustrated in FIG. 2 includes those components described above with reference to FIG. 1 which implement the transcutaneous RF link.

Stimulus controller 208 preferably receives the selected maxima from the audio signal analysis block 206 and determines, based on the stimulation strategy being implemented, information and signals for use in applying stimulus via the electrode array 142. For example, stimulus controller 208 may select for each of the received maxima the electrode(s) to be used, the timing, the mode of stimulation, and the amplitude of the stimulation to be applied. The selected mode of stimulation may be, for example, bi-polar or mono-polar. In addition, the desired electrodes which are to electrically coupled in accordance with the teachings of the present invention may be specified by stimulus controller 208. For example, the electrodes may be grouped based on a pre-defined strategy for grouping the electrodes (e.g., a strategy based on testing of the implant system after implantation in an implant recipient), or, for example, stimulus controller 208 may dynamically determine how to group electrodes based on, for example, characteristics of the received maxima, or the electrodes may be grouped based on some combination of both predefined information and dynamic information. Various exemplary strategies for grouping electrodes are discussed in further detail below.

There are a myriad of techniques which may be implemented to effect the requisite communications between stimulus controller 208 and output switch controller 210 to cause output switch controller 210 to electrically couple selected electrodes as described herein. These include, but are not limited to, sending commands specifying specific groupings of electrodes, identifying entries of a table stored in output switch controller 210 which stores various electrode grouping options, etc. As one of originally skill in the art would appreciate, the implemented communication technique depends on a variety of factors including the particular implementation of output switch controller 210. It should be appreciated that any communication technique now or later developed may be implemented in embodiments of the present invention. In one embodiment, the above mode information includes and indication of, for example, double electrode mode (i.e., group 2 electrodes together for each stimulus application), triple electrode mode (i.e., group 3 electrodes together for each stimulus application), a custom grouping of electrodes, etc.

Stimulus controller 208 provides the determined mode, timing, and electrodes (e.g., grouping) information to output switch controller 210, and provides the determined amplitude information to a stimulus current generator 212. Output switch controller 210 and stimulus current generator 212 then use the received information to stimulate electrodes 202 of electrode array 142. A further description of methods and systems for stimulating the electrodes of electrode array 142 according different modes of stimulation (e.g., mono-polar and bi-polar stimulation) is provided in further detail below. Output switch controller 210 and stimulus current generator 212 may be included for example in the stimulator unit 120 of FIG. 1.

The following provides a more detailed description of exemplary methods and systems for delivering stimulus to an electrically coupled group of two or more electrodes in a cochlear implant system. As discussed in further detail below, two or more electrodes are directly or indirectly electrically coupled to each other so that a stimulating signal may be simultaneously applied to the electrically-coupled electrodes by a single current source. It should be appreciated by those of ordinary skill in the art, however, that the teachings of the present invention may be readily applied to any cochlear implant, prosthetic hearing device or medical stimulating device now or later developed.

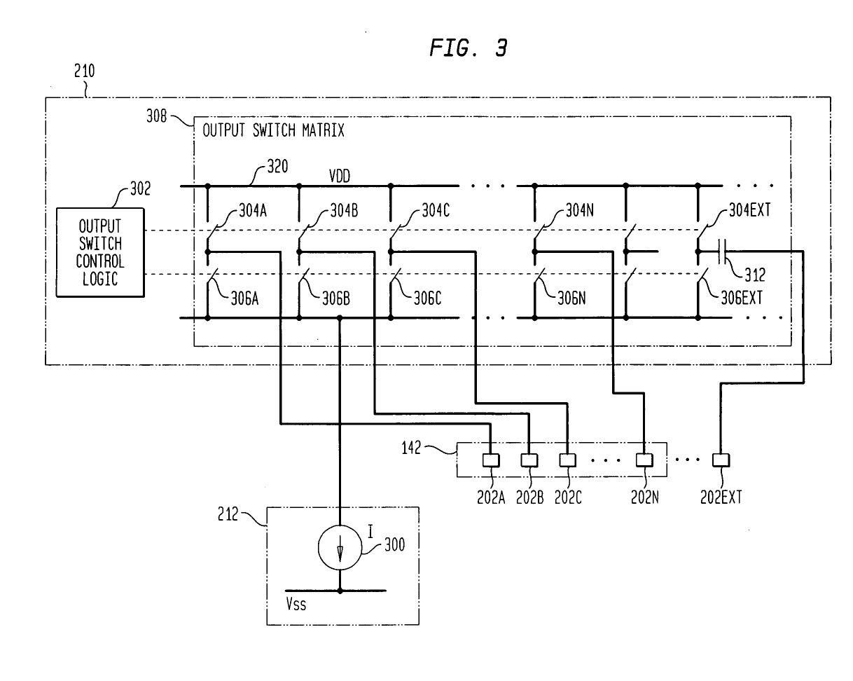

FIG. 3 is a schematic block diagram of an exemplary embodiment of output switch controller 210. This embodiment of output switch controller 210 implements an electrode stimulation circuit for mono-polar stimulation. In this exemplary embodiment, output switch controller 210 comprises an output switch matrix 308 controlled by output switch control logic 302.

As illustrated, each electrode 202A, 202B, 202C, . . . 202N (collectively and generally referred to as electrode or electrodes 202) of electrode array 142 is connected to a voltage VDD 320 via an associated switch 304A, 304B, 304C, . . . 304N, respectively, (collectively and generally referred to as electrode or switch or switches 304). Each electrode 202A, 202B, 202C, . . . 202N is also connected to stimulus current generator 212 via an associated second switch 306A, 306B, 306C, . . . 306N, respectively, (collectively and generally referred to as electrode or switch or switches 306).

Electrode assembly 118 also comprises an additional, extra-cochlea electrode 202EXT similarly connected to voltage source 320 and current source 212 via corresponding switches 304EXT and 306EXT, respectively via a capacitor 312. As one of ordinary skill in the art would understand, extra-cochlea electrode 202EXT is utilized for use in mono-polar stimulation.

Output switch control logic 302 is connected to and controls switches 304 and 306 for each electrode 202 of electrode array 104 as well as switches 304EXT and 306EXT for electrode 202EXT.

Further, although a single dotted line is illustrated as connecting switches 304 and 308 to out switch control logic 322 and a single dotted line is illustrated as connecting switches 306 and 310 to output switch control logic 322, one of skill in the art would be aware that this illustration is a simplified logical diagram and that in practice each of these connections may be a separate direct connection between switches 304 and 306 and output switch control logic 302. The functions and operations performed by output switch control logic 302 are described in detail below.

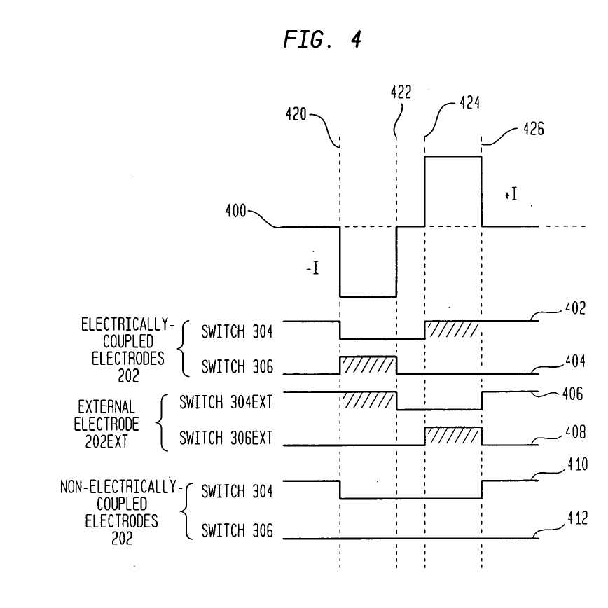

FIG. 4 includes a series of timing diagrams illustrating the manner in which output switch control logic 302 controls output switch matrix 308 in accordance with one embodiment of the present invention. In FIG. 4, stimulating signal 400 is applied to cochlear 116 via an electrically-coupled electrodes 202. Because both a positive and negative currents are applied in succession, as illustrated in FIG. 4, stimulating signal 400 is a biphasic waveform. In the exemplary embodiment shown in FIG. 4, a negative phase of biphasic stimulating signal 400 occurs prior to a positive phase, and there is an inter-pulse gap occurring between the negative and positive pulses of stimulating signal 400.

For explanatory purposes, those electrodes 202 which are to be electrically coupled to form a single group of electrodes are collectively referred to herein as “Electrode Group 1.” Timing diagrams 402 and 404 illustrate the timing for switches 304 and 306, respectively, of electrodes 202 of Electrode Group 1. Timing diagrams 406 and 408 illustrates the state of switches 304EXT and 306EXT which, as noted, are associated with electrode 202EXT. In each of these timing diagrams, a high logic level indicates that the switch is closed and a low logic level indicates that the switch is open.

In this example, prior to receipt of a stimulus signal, all switches 304 and 304EXT are closed to connect each electrodes 202 and 202EXT, respectively, to voltage source 320. This is illustrated by the high logic levels of timing diagrams 402 and 406 prior to the time represented by dashed line 420. Also, switches 306 and 306EXT are open to disconnect each electrode 202 and 202EXT, respectively, from stimulus current generator 212. This is illustrated by the low logic levels of timing diagrams 404 and 408 prior to the time represented by dashed line 420.

Thereafter, output switch control logic 302 receives a stimulation signal directing output switch control logic 302 to stimulate all electrodes in Electrode Group 1 to simultaneously deliver a stimulation signal to electrodes 202 of Electrode Group 1. In response, output switch control logic 302 controls switches 304 of Electrode Group 1 to open to disconnect the selected electrodes 202 from voltage source 320, as shown by waveform 402 transitioning from a high logic level to a low logic level at the time marked by dashed line 420 in FIG. 4.

As shown by timing diagrams 410 and 412, switches 304 and 306 associated with electrodes 202 which are not included in Electrode Group 1 are transitioned to tile open state during stimulation of Electrode Group 1. As such, in this example, when switches 304 of Electrode Group 1 transition to the low logic level, all switches 304 and 306 for electrodes 202 other than extra-cochlea electrode 202EXT transition to, or remain in, a low logic level, as illustrated by timing diagrams 410 and 412 at dashed line 420. This results in the phase of stimulating signal 410 between dashed lines 420 and 422 to be applied to cochlear 116 via electrodes 202 included in Electrode Group 1. As illustrated by stimulating signal 410, this permits current to flow from extra-cochlea electrode 202EXT to electrically coupled electrodes 202 of Electrode Group 1 resulting in application of a negative current, “−I” to electrodes 202 of Electrode Group 1.

To form an inter-pulse gap of stimulating signal 410 between dashed lines 422 and 424; that is, the portion of stimulating signal 410 which occurs between the positive and negative phases of stimulating signal 410, output switch control logic 302 performs the following operations.

Switch 304 of the electrically-coupled electrodes 202 is maintained in a low logic level, while switch 306 of the electrically-coupled electrodes 202 is transitioned from a high logic level to a low logic level. Conversely, switch 306 of the extra-cochlear electrode 202EXT is maintained in a low logic level, while switch 304 of the extra-cochlear electrode 202EXT is transitioned from a high logic level to a low logic level. This causes the negative phase of stimulating signal 410 to cease at the time represented by dashed line 422, and to remain in this state until the time represented by dashed line 424 occurs.

To form a positive pulse between the time represented by dashed lines 424 and 426, the following operations are performed by output switch control logic 302. Output switch control logic 302 controls switches 304, 304EXT, 306 and 306EXT to open and close such that current flows in the opposite direction; that is, from electrodes 202 of Electrode Group 1 to extra-cochlea electrode 202EXT. More specifically, switch 304 associated with electrodes 202 of Electrode Group 1 are closed. This is depicted in FIG. 4 by timing diagram 402 transitioning from a low logic level to a high logic level at the time represented by dashed line 424. Switches 306 corresponding to the electrically-coupled electrodes 202 are maintained at a low logic level, as illustrated by timing diagram 404.

Similarly, switch 306EXT associated with extra-cochlear electrode 202EXT is closed to connect extra-cochlear electrode 202EXT to stimulus current generator 212. This is depicted in FIG. 4 by timing diagram 408 transitioning from a low logic level to a high logic level at the time represented by dashed line 424, and maintained at the high logic level until the time represented by dashed line 426. Switch 304EXT associated with extra-cochlear electrode 202EXT is opened to disconnect extra-cochlear electrode 202EXT from voltage source 320. This is depicted in FIG. 4 by timing diagram 406 being maintained in a low logic level from the time represented by dashed line 424 to the time represented by dashed line 426. This permits current to flow from electrodes 202 of Electrode Group 1 to extra-cochlear electrode 202EXT resulting in application of a positive current, “+I,” to cochlear 116 via electrodes 202 of Electrode Group 1, as illustrated by timing diagram 410. Switches 304 and 306 of non-electrically coupled electrodes 202 are maintained in their open state from the time represented by dashed line 424 and the time represented by dashed line 426, as illustrated by timing diagrams 410 and 412 in FIG. 4.

After application of the positive stimulus for a fixed period of time, switches 304, 304EXT, 306 and 306EXT transition to the state they were in prior to application of the stimulus to await the next stimulus. This transition is illustrated at dashed line 426.

As one of ordinary skill in the art would find apparent, the quantity of electrodes 202 which are electrically coupled to each other may be an quantity necessary for a particular application or recipient. This is described in further detail below. It should also be appreciated that although the above embodiment was described with reference to bi-phasic current simulation, in other embodiments, other types of current stimulation may be used, such as, for example, asymmetric current stimulation (e.g., the first and second phases have different amplitudes and time durations), tri-phasic current stimulation, non-rectangular types of current stimulation, etc.

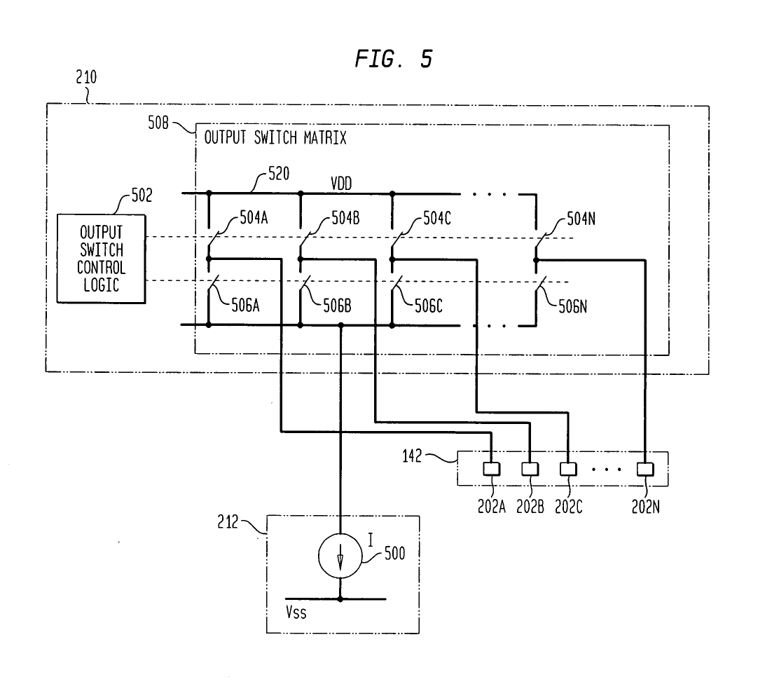

FIG. 5 is a schematic block diagram of an embodiment of output switch controller 210 that implements bi-polar stimulation. This diagram is very similar to the diagram of FIG. 3 with the exception that it does not include an extra-cochlea electrode 202EXT or associated capacitor 312. Rather, one or more electrodes 202 of electrode array 142 is/are utilized the complete the current path for the stimulus current signal applied via a plurality of electrically-coupled electrodes 202. U.S. Pat. No. 4,532,930, the entire contents and disclosure of which is hereby incorporated by reference, describes suitable electrode switching schemes implementing bipolar stimulation.

As one of ordinary skill in the art will understand, in the above embodiments the electrical coupling of selected electrodes 202 is indirect through the voltage and current sources 320 and 300, respectively. It should be appreciated, however, that in alternative embodiments, the selected electrodes 202 may be electrically coupled directly to each other. It should also be appreciated that in still other embodiments, various circuit components may be utilized to provide or support the electrical coupling, for example, to achieve desired electrical characteristics of the group of electrically-coupled electrodes 202. These and other alternatives to directly or indirectly electrically couple a selected plurality of electrodes are within the scope of the present invention.

To determine which electrodes 202 are to be electrically coupled to each other, an audiologist or other competent party may conduct sound frequency mapping to neurons in cochlea 116 using a diagnostic programming system, or other suitable technique. The audiologist would be interested in electrodes that are aberrant in pitch percept, impedance or C-level, i.e. any behaviour that may indicate the beneficial application of wider electrodes. The frequency mapping information may then be programmed into speech processor 126 to provide proper signals to the desired electrodes 202 of one or more electrically-coupled groups of electrodes.

In certain embodiments of the present invention, the total number of electrodes 202 in electrode array 142 is a fixed number, such as 22, or may be a custom number of more or less electrodes depending on the particular situation.

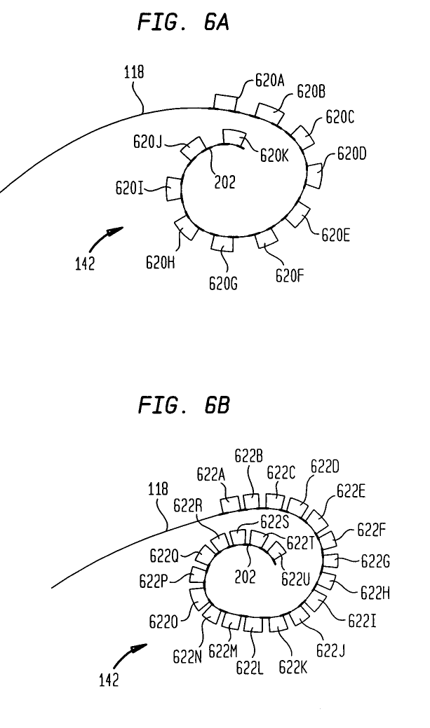

FIGS. 6A-6D are schematic illustrations of exemplary approaches in which electrodes of electrode array 142 with 22 electrodes 202 may be electrically coupled to each other in accordance with embodiments of the present invention.

In FIG. 6A, electrodes 202 of electrode array 142 are electrically coupled to form eleven groups 620A-620K each having two electrodes 202 electrically coupled to any other. No electrodes 202 are shared among electrode groups 620. Thus, in this example, the present invention is implemented to form 11 independent stimulation channels out of the 22 electrodes 202 in electrode array 142. In contrast, in FIG. 6B, neighboring electrodes 202 of electrode array 142 are electrically coupled such that each electrode group 622 shares an electrode 202 with its adjacent electrode group(s) 620. Thus, in this example, the present invention is implemented to form 21 stimulation channels out of the 22 electrodes 202 in electrode array 142. Similarly, if each electrode group contains three electrodes 202, the electrode groups can comprise independent electrodes 202, in which case there would be seven independent triple electrode groups, or the electrode groups can comprise shared electrodes 202, in which case there would be 20 shared triple electrode groups.

As shown in FIG. 6C, electrodes 202 of electrode array 142 are electrically coupled to form two groups of three electrode (electrode groups 602A and 602B), two groups of two electrodes (electrode groups 604A and 604B) and 12 single electrodes 202 which are not electrically coupled to any other electrode 202 and, therefore are not members of an electrode group. Thus, in this example, the present invention is implemented to form 16 independent stimulation channels out of the 22 electrodes 202 in electrode array 142.

Another examiner is illustrated in FIG. 6D. There, electrodes 202 of electrode array 142 are electrically coupled to provide two groups of four electrodes each (electrode groups 606A and 606B), two groups of three electrodes each (electrode groups 608A and 608B) and four groups of two electrode (electrode groups 610A, 610B, 610C, and 610D). Thus, in this example, the present invention is implemented to form 8 independent stimulation channels out of the 22 electrodes 202 in electrode array 142. It should be understood that these are but two exemplary embodiments and that aspects of the present invention may be implemented to provide other electrode geometries.

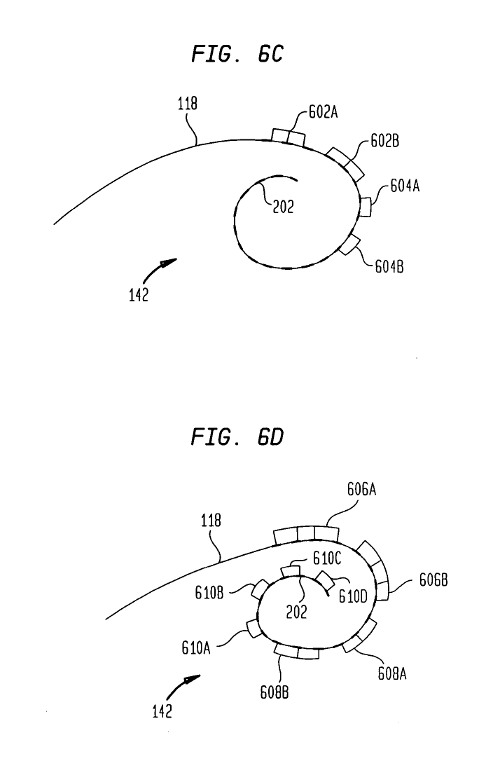

As discussed above, in some embodiments a group of electrically-coupled electrodes 202 includes a plurality of adjacent electrodes. During application of a stimulus, such adjacent electrodes deliver a common stimulus. FIG. 7 is a graph illustrating the relative impedance of two groups of electrically-coupled electrodes 202 and a single electrode 202. As show, the impedance of two electrically-coupled electrodes 202 is approximately 35% less than the impedance of a single electrode 202, and the impedance of three electrically-coupled electrodes 202 is approximately 50% less than the impedance of a single electrode 202.

Thus, one advantage of electrically coupling a plurality of electrodes 202 is that the electrically-coupled group of electrodes has a collective impedance which is substantially less than the impedance of a single electrode. Because power consumption typically increases with increasing stimulus current, a reduction in electrode impedance reduces the power consumption of the stimulating medical device. This is particularly advantageous when used in conjunction with high-density electrode arrays such as electrode array 142.

It is apparent from the present embodiments that an increase in electrode surface area may compensate for a variety of “non-ideal” conditions occurring with intra-cochlea electrode arrays. For example, as noted, increased area results in wider current spread and reduces impedance. An electrode system that selectively allows area to be adapted to a specific cochlea, therefore has many advantages. One advantage of this approach is that it may be beneficial to combine electrodes to intentionally increase the spread of excitation over a region of poor neural survival and at the same time reduce the impedance, which would help to compensate for the higher stimulation currents that may be required for such sites.

Yet another benefit of multiple electrode modes is to emulate the way in which the area of excitation in the acoustically stimulated cochlea increases with increasing sound intensity using a single stimulus. Present implant systems rely on increasing the stimulus amplitude on a single electrode to map increasing loudness, or alternatively, stimulating several electrodes simultaneously or in rapid succession, requiring the processing of multiple stimuli. Addressing multiple electrodes requires more system bandwidth and power, whereas electrically-coupling multiple electrodes adapts the electrode array on a per stimulus basis, without impacting bandwidth or power.

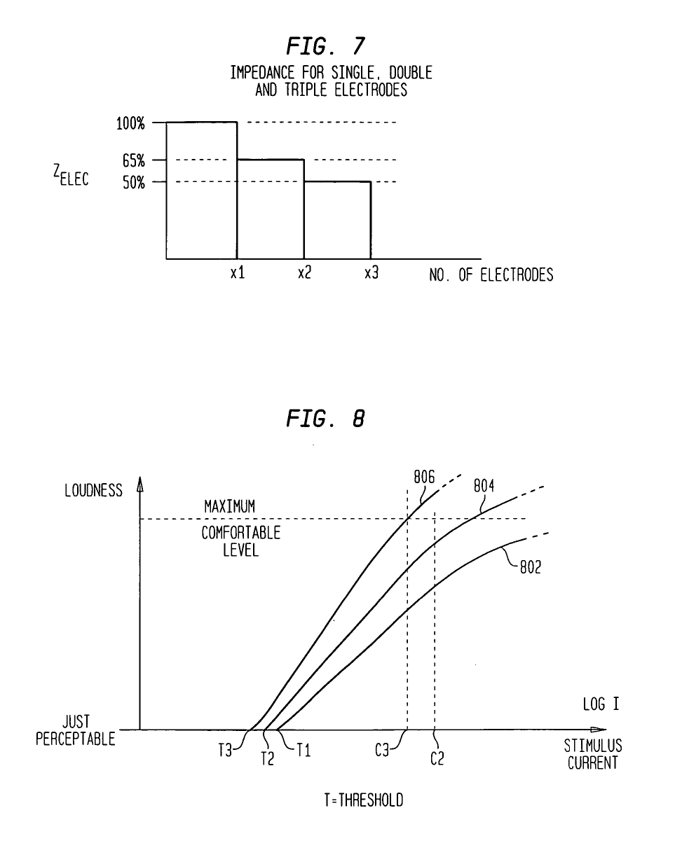

FIG. 8 illustrates exemplary loudness growth functions for single, double and triple electrode groups. In this figure, the same stimulus parameters, e.g., rate and width, etc. of stimulation were used. Curve 802 illustrates an exemplary loudness growth function for a single electrode, curve 804 illustrates an exemplary loudness growth function for a group of two electrically-coupled electrodes, and curve 806 illustrates an exemplary loudness growth function for a group of three electrically-coupled electrodes. As illustrated, the larger the group of electrodes the greater the perceived loudness for a common stimulus current. For example, as illustrated by curve 802 a single electrode may be unable to reach an implant's recipients maximum comfort level, while grouping that electrode with one or two other electrodes may permit the maximum comfort level to be reached, as illustrated by curves 804 and 806.

Previously, it was expected that positioning the electrode closer to the modiolus (the inner wall of the cochlea) would reduce the stimulation charge requirements; however, in actuality, as the electrode gets closer to the modiolus, the degree of current spread reduces. This has the effect of reducing the perceived loudness, necessitating a further increase in stimulus current or total stimulation rate to achieve C-level (maximum stimulation level not causing discomfort). Embodiments of the present invention may be used to help minimize or eliminate these effects due to an electrode array being positioned close to the modiolus. For example, by grouping multiple electrodes, current spread may be broadened to, for example, stimulate more neurons in order to achieve a louder sound across a particular frequency range.

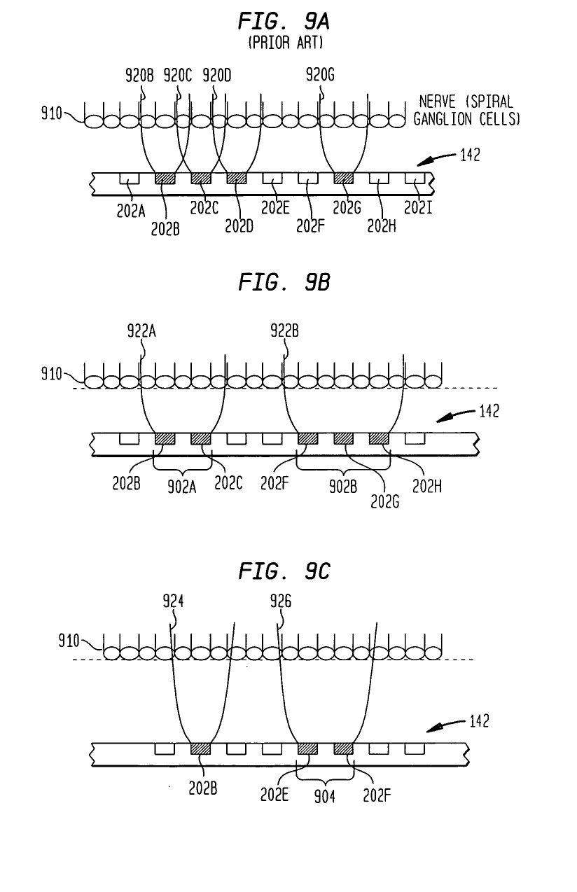

FIGS. 9A-9C illustrate various exemplary representations of current spread for different stimulation schemes. FIG. 9A illustrates an example of a conventional electrode geometry in which no electrodes 202 are electrically coupled to each other, and the electrodes are positioned close to modiolus 910 as compared to the relative positioning shown in FIG. 9C. As shown in FIG. 9A, the spread of excitations, 920B, 920C and 920D, for electrodes 202B, 202C, and 202D, respectively, have relatively little overlap due to close proximity of the electrodes to the modiolus.

FIG. 9B illustrates an exemplary embodiment which two groups of electrodes 902A and 902B are individually stimulated but the electrodes 202 in each group are simultaneously stimulated since they are electrically coupled in accordance with the teachings of the present invention. As with the example of FIG. 9A, in this example the electrodes are likewise positioned close to modiolus 910 as compared with the arrangement shown in FIG. 9C. As shown, the spread of excitation 922A for two-electrode group 902A is less than the spread of excitation 922B for three-electrode group 902B. Also, because the electrodes in each group are simultaneously stimulated, they provide a wider spread of excitation (922A and 922B) than if only a single electrode was stimulated.

FIG. 9C is a diagrammatic illustration of the current spread occurring in the auditory nerves 910 in response to a stimulation signal applied to a single electrode 202B and to an electrode group 904 of two electrically-coupled electrodes 202E and 202F on an array 142 positioned further away from the modiolus as compared with the arrangements shown in FIGS. 9A and 9B.

FIG. 9C illustrates an exemplary embodiment in which a single electrode 202B and a single group of electrodes 904 are stimulated. In this example the electrodes are positioned further from modiolus 910 than as illustrated in FIGS. 9A and 9B. As shown in FIGS. 9C, as a result of being further from modiolus 910, the spread of excitation 926 for the group of electrodes 904 and the spread of excitation 924 for single electrode 202B are both wide, and spread of excitation 926 is only marginally wider than spread of excitation 924.

Various embodiments of present invention are (1) better able to adapt to non-ideal neural survival in cochlea; (2) able to flexibly configure a high density array by utilizing combinations of single and variable width electrodes; (3) able to reduce electrode impedance, which translates to reduced implant power consumption and lower battery power in the external speech processor, which is particularly advantageous for BTE speech processors; and (4) able to selectively broaden current spread in a high-density electrode array to emulate spread of excitation for loud sounds. Further, embodiments of the present invention may be incorporated into present and future implant designs and provide a simple means for realizing the benefits detailed above.

The grouping of electrodes in electrode array 142 may be determined prior to implantation or may be dynamically determined during use of the stimulating medical device in which the invention is implemented. For example, in certain embodiments, stimulus controller 208 may determine to increase or decrease the number of electrodes 202 in a group based on characteristics of a received audio signal. These characteristics may include, for example, the loudness of the signal in the frequency range for the group, and/or other factors. For example, if a received audio signal's amplitude (loudness) is high in the frequency range of the electrode group (e.g., it exceeds a threshold), stimulus controller 208 may increase the size of the group, such as, for example, from one electrode to two or more electrodes. Or in other examples, multiple electrodes may be electrically-coupled or -decoupled to achieve a desired spread of excitation (SOE).

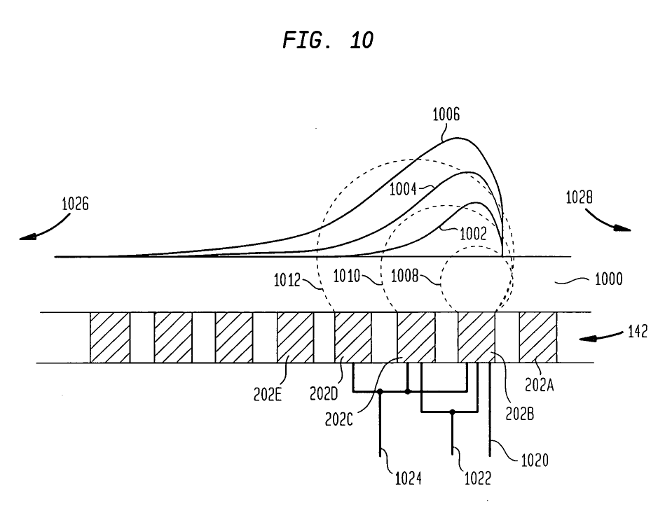

FIG. 10 is a diagram illustrating how the natural increase in spread of excitation for an acoustically excited cochlea may be emulated electrically via stimulation of a group of electrodes in accordance with embodiments of the present invention. As shown, curves 1002, 1004, and 1006 illustrate how the spread of excitation occurs in a normal cochlea 116 in response to the application to region 1008 of an auditory nerve 1000 via a single electrode 202B of stimulating signals having successively increasing signal amplitudes. Note that the spread of excitation occurs in a basal direction 1026 from the peak excitation point. That is, the spread of excitation involves more of the high frequency neurons but does not spread significantly in an apical direction 1028 (i.e. towards the low frequency end of cochlea 116).

To emulate the spread of excitation represented by curve 1004, two electrodes 202B and 202C may be electrically coupled in accordance with the teachings of the present invention. Such electrical coupling is represented schematically by lead 1022 connected to both electrodes 202B and 202C. The targeted region of auditory nerve 1000 stimulated by application of a stimulus via the electrically-coupled electrodes 202B, 202C is depicted by dashed line 1010. The resulting current spread of such a stimulation would be similar to that represented by curve 1004.

Similarly, to emulate the spread of excitation represented by curve 1006, three electrodes 202B, 202C and 202D may be electrically coupled in accordance with the teachings of the present invention. Such electrical coupling is represented schematically by lead 1024 connected to electrodes 202B, 202C and 202D. The targeted region of auditory nerve 1000 stimulated by application of a stimulus via the electrically-coupled electrodes 202B, 202C, 202D is depicted by dashed line 1012. The resulting current spread of such a stimulation would be similar to that represented by curve 1006.

As such, a received audio signal may be analyzed to determine information regarding the desired spread of excitation in application of the stimulus. This determined spread of excitation information may then be considered when selecting the number of electrodes 202 to electrically couple for application of the stimulus. It should be noted that in other embodiments, more accurate spread of excitation control may be achieved using more complex modes of stimulation, at the cost of complexity and power (and bandwidth).

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

All documents, patents, journal articles and other materials cited in the present application are hereby incorporated by reference. Although the present invention has been fully described in conjunction with several embodiments thereof with reference to the accompanying drawings, it is to be understood that various changes and modifications may be apparent to those skilled in the art. Such changes and modifications are to be understood as included within the scope of the present invention as defined by the appended claims, unless they depart therefrom.

What is claimed is:

1. A method for delivering a stimulating signal by a stimulating medical device having a plurality of electrodes, comprising:
electrically coupling a first set of at least two of the plurality of electrodes; and
simultaneously delivering to the first set of electrically-coupled electrodes a stimulation signal suitable for application to a target tissue of a recipient.

2. The method of claim 1, wherein simultaneously delivering to the first set of electrically-coupled electrodes a stimulation signal comprises:
simultaneously delivering a stimulation signal to the first set of electrically-coupled electrodes via a single current source.

3. The method of claim 1, further comprising:
delivering a stimulation signal to an individual electrode, wherein the stimulation signal delivered to the an individual electrode is delivered at a different time than the stimulation signal delivered to the first set of electrically-coupled electrodes.

4. The method of claim 3, further comprising:
sequentially delivering stimulating signals to the individual electrode and the first set of electrically-coupled electrodes in accordance with a stimulation strategy.

5. The method of claim 1, further comprising:
forming a plurality of additional sets of electrically-coupled electrodes; and
simultaneously delivering to at least one of the additional sets of electrically-coupled electrodes, a stimulation signal suitable for application to the target tissue of the recipient.

6. The method of claim 1, wherein simultaneously delivering to the first set of electrically-coupled electrodes a stimulation signal comprises:
closing a first switch corresponding to a first electrode of the first set of electrically-coupled electrodes;
closing a second switch correspond to a second electrode of the first set of electrically-coupled electrodes, wherein the first switch and the second switch are close such that both the first and second electrodes are simultaneously connected to a common current source.

7. The method of claim 6, wherein when both the first and second switches are closed current flows between the first and second electrodes and an extra-cochlea electrode.

8. The method of claim 1, further comprising:
transmitting the stimulation signal via a transcutaneous link to a receiver implanted in the recipient.

9. The method of claim 1, further comprising:
determining the electrodes comprising the first set of electrically-coupled electrodes based on one or more characteristics of the recipient.

10. The method of claim 9, wherein the one or more the characteristics of the recipient are determined based on one or more tests conducted after implantation of the medical device in the recipient.

11. The method of claim 1, wherein the stimulating medical device is a prosthetic hearing implant, and wherein the method further comprises:
receiving an acoustical signal; and
determining the stimulation signal based on the received acoustical signal.

12. The method of claim 11, further comprising:
determining the electrodes comprising the first set of electrically-coupled electrodes based on one or more characteristics of the received acoustical signal.

13. The method of claim 12, wherein the one or more characteristics of the received acoustical signal include a loudness of the acoustical signal in a frequency range corresponding to the electrodes in the first set of electrodes; and
wherein the determining the electrodes comprises determining the number of electrodes in the first set of electrically-coupled electrodes based on the loudness.

14. A method for stimulating a recipient comprising:
disposing a plurality of tissue-stimulating electrodes in a physical arrangement on or in the recipient; and
adjusting a geometry of the plurality of electrodes without replacing or altering the physical arrangement of the plurality of electrodes.

15. The method of claim 14, wherein adjusting a geometry of the plurality of electrodes comprises:
forming at least one electrode group each comprising two or more of the plurality of electrodes electrically coupled to each other; and
for each of the at least one electrode group, simultaneously delivering to the two or more electrically-coupled electrodes of each of the at least one electrode group, a stimulation signal via a single current source.

16. The method of claim 15, wherein the method further comprises:
delivering stimulating signals to any individual electrodes and the at least one electrode group in accordance with a selected stimulation strategy.

17. The method of claim 16, wherein delivering stimulating signals to only individual electrodes and the at least one electrode group in accordance with a selected stimulation strategy comprises:
sequentially delivering stimulating signals to any individual electrodes and the at least one electrode group in accordance with a selected stimulation strategy.

18. The method of claim 15, wherein forming at least one electrode group each comprising two or more of the plurality of electrodes electrically coupled to each other comprises:
forming a plurality of the electrode groups such that at least two of the electrode groups share at least one electrode.

19. The method of claim 15, wherein forming at least one electrode group each comprising two or more of the plurality of electrodes electrically coupled to each other comprises:
forming a plurality of the electrode groups such that at least two of the electrodes of at least one of the electrode groups are adjacent to each other.

20. The method of claim 15, wherein the tissue-stimulating electrodes are included as part of a prosthetic hearing implant, and wherein the method further comprises:
receiving an acoustical signal; and
determining the stimulation signal based on the received acoustical signal.

21. The method of claim 20, further comprising:
determining the electrodes forming the at least one electrode group based on one or more characteristics of the received acoustical signal.

22. A cochlear implant system, comprising:
a plurality of electrodes disposed in a cochlear of a recipient;
a speech processor for processing received acoustical signals; and
a stimulator unit, responsive to the speech processor, configured to electrically couple selected electrodes and to simultaneously deliver a stimulation signal to the electrically-coupled electrodes via a stimulus current generator.

23. The system of claim 22, wherein the stimulator unit comprises:
a stimulus controller configured to select two or more electrodes from the plurality of electrodes; and
all output switch controller configured to electrically couple the selected electrodes to simultaneously deliver a stimulation signal to the electrically-coupled electrodes via a stimulus current generator.

24. The system of claim 23, wherein the output switch controller comprises:
an output switch matrix comprising a plurality of first switches to connect each said electrode to a voltage source and a plurality of second switches to connect each said electrode to the stimulus current generator; and
output switch control logic configured to control said first and second switches to simultaneously generate a stimulation signal on the selected electrodes thereby electrically-coupling the selected electrodes.

25. The system of claim 22, wherein the stimulator unit is further configured to deliver a stimulation signal to an individual electrode, wherein the stimulation signal delivered to the an individual electrode is delivered at a different time than the stimulation signal delivered to the electrically-coupled electrodes.

26. The system of claim 25, wherein the stimulator unit is further configured to sequentially deliver the stimulating signals to the individual electrode and the first set of electrically-coupled electrodes in accordance with a stimulation strategy.

27. The system of claim 22, wherein the stimulator unit is further configured to form a plurality of additional sets of electrically-coupled electrodes and simultaneously deliver to at least one of the additional sets of electrically-coupled electrodes, a different stimulation signal via the stimulus current generator.

28. The system of claim 22, further comprising an extra-cochlea electrode.

29. The system of claim 22, further comprising:
a receiver implanted under the skin of the recipient; and
a transmitter configured to transmit the stimulation signal via a transcutaneous link to the receiver.

30. The system claim 22, wherein the electrically-coupled electrodes are adjacent to each other.

31. The system of claim 22, wherein the electrodes comprising the electrically-coupled electrodes are determined based on one or more characteristics of the recipient.

32. The system of claim 31, wherein one or more of the characteristics of the recipient are determined based on one or more tests conducted after disposition of the plurality of electrodes in the recipient.

33. The system of claim 22, wherein the stimulator unit is further configured to determine the electrodes comprising the electrically-coupled electrodes based on one or more characteristics of the received acoustical signal.

34. The system of claim 33, wherein the one or more characteristics of the received acoustical signal include a loudness of the acoustical signal in a frequency range corresponding to the electrodes comprising the electrically-coupled-electrodes; and
wherein the stimulator unit is configured to determine the number of electrodes in the first set of electrodes based on the loudness of the acoustical signal in the frequency range.

35. A system for delivering a stimulating signal by a stimulating medical device having a plurality of electrodes, comprising:
means for electrically coupling a first set of at least two of the plurality of electrodes; and
means for simultaneously delivering to the first set of electrically-coupled electrodes a stimulation signal suitable for application to a target tissue of a recipient.
